(19) United States
(12) Patent Application Publication
(10) Pub. No.: US 2014/0323989 A1
(43) Pub. Date: Oct. 30, 2014

(54) WEARABLE INFUSION DEVICE WITH LOW PROFILE HANDLE

(71) Applicant: CALIBRA MEDICAL, INC., Redwood City, CA (US)

(72) Inventors: Daniel L. Baker, Drexel Hill, PA (US); Brett Cross, Redwood City, CA (US); John McKenzie, San Carlos, CA (US)

(73) Assignee: Calibra Medical, Inc., Redwood City, CA (US)

(21) Appl. No.: 13/869,837

(22) Filed: Apr. 24, 2013

(51) Int. Cl. A61M 5/158

(52) U.S. Cl. CPC A61M 5/158
USPC 604/257

(57) ABSTRACT

The invention provides infusion devices that utilize a low profile handle. The handle is useful in retaining a needle in, and removing the needle from, the device.

### FIELD OF THE INVENTION

The present invention relates to medication infusion devices. In particular, the invention relates to infusion devices that enable liquids to be conveniently and safely self-administered.

### BACKGROUND OF THE INVENTION

The administration of medications for chronic diseases, such as the administration of insulin for the treatment of people with diabetes, traditionally was accomplished using a syringe. More recently, pen-like injection devices have been used for this purpose. Both of these forms of administration require the medication recipient to be punctured with a needle to deliver the medication. If the recipient needs multiple, daily injections of medication, this results in the recipient being punctured with a needle multiple times in a day.

More recently, durable medication delivery pumps have been developed that deliver medication stored in a reservoir within the device through tubing and a catheter. The catheter is inserted into the user of the device and is changed only once every several days. These pumps decrease the number of times the user must be punctured by a needle, but they are expensive to manufacture, complex to operate, cumbersome to use, and require significant amounts of training of the user. Additionally, such devices require tubing from the device to the catheter site located on the pump user's body.

More cost-effective and simple disposable medication infusion devices have been developed whereby a small injection or pump system, that eliminates the need for tubing, is attached directly to the user's skin. The devices deliver medication into the user by manual or automatic pumping of small doses of medication out of an in-dwelling cannula that is a part of the infusion device. As a result, a medication, such as insulin, may be carried by the user discreetly and conveniently administered in multiple doses with only one needle puncture of the user required every several days. Examples of such devices are disclosed in co-pending, U.S. application Ser. No. 13/737,859, which is hereby incorporated in its entirety herein by reference.

The present invention provides an infusion device that improves safety and convenience for the user in handling of the needle used with the device.

### BRIEF DESCRIPTION OF THE DRAWINGS

FIG. 1 is a top perspective view of a drug infusion system of the invention.

FIG. 1A is a bottom perspective view of a needle handle useful in the system shown in FIG. 1.

FIG. 1B is a top perspective view of the needle handle shown in FIG. 1A in the deployed position.

FIG. 2 is an exploded perspective view of the system of FIG. 1.

FIG. 3 is a bottom view of the system of FIG. 1.

FIG. 4 is an exploded perspective view of the system of FIG. 3.

FIG. 5 is a perspective view illustrating a manner of filling the reservoir of the system of FIG. 1.

FIG. 6 is an exploded view illustrating the removal of a cannula cover of the system of FIG. 1.

FIG. 7 is a side perspective view illustrating the device of the system of FIG. 1.

FIG. 8 is a side perspective view, illustrating the device of the system of FIG. 1 after deployment and during removal of a needle and needle handle.

FIG. 9 is a side perspective view illustrating the safe storage of the needle in the cannula cover.

FIG. 10 is a perspective view of the device of the system of FIG. 1 with a top cover removed to illustrate the internal components of the device.

FIG. 11 is partial perspective view illustrating the last-dose lock-out and occlusion detection mechanism of the device of FIG. 1.

FIG. 12 is a sectional view of the device of the system of FIG. 1 illustrating internal components of the device.

FIG. 13 is a magnified, perspective view showing an internal section of the device.

FIG. 14 is an exploded, perspective view of a portion of the device showing selected internal components.

FIG. 15 is a magnified, perspective side view with portions cut-away of a cannula and an insertion needle.

FIG. 16 is a partial, side view in perspective showing the syringe, needle bushing and needle during the filling of the device.

FIG. 17 is a side view of the device of the system of FIG. 1 showing an alternative embodiment of the needle handle.

FIG. 18 is a top view of the system of FIG. 17.

FIG. 19 is a side view of the handle of FIG. 17 in use.

FIG. 20 is a top perspective view of another alternative embodiment of a handle of the invention.

FIG. 21 is a side perspective view of the handle of FIG. 20 in the deployed state.

FIG. 22 is a side perspective view of the handle of FIG. 20 in the closed state.

FIG. 23 is a side perspective view of the bottom of the handle of FIG. 20.

### DETAILED DESCRIPTION OF THE INVENTION AND PREFERRED EMBODIMENTS

In general, the invention provides infusion devices, for example devices for administration of a dose of medication, such as insulin, to a device user. The invention may find particular utility in administering predetermined volumes of insulin while the device is being worn on the skin for a period of time, for example up to several days. When a dose of medication is desired, the device is activated to provide the desired dose to the user.

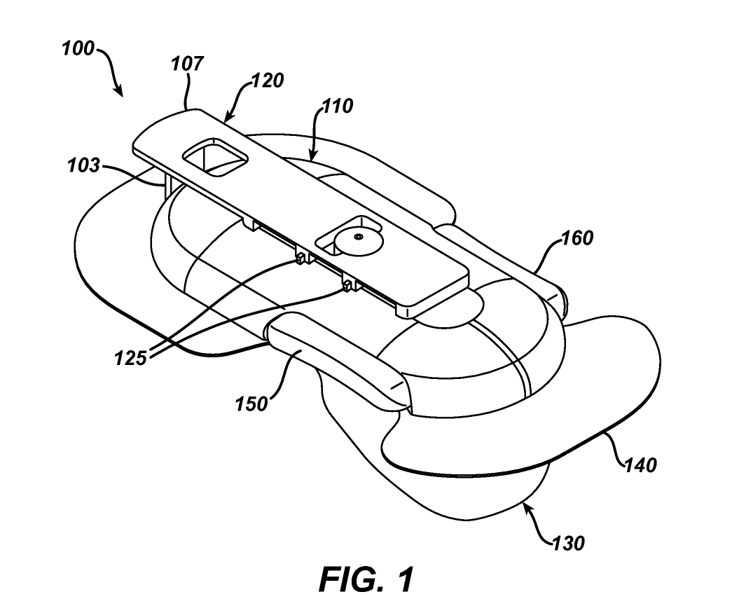

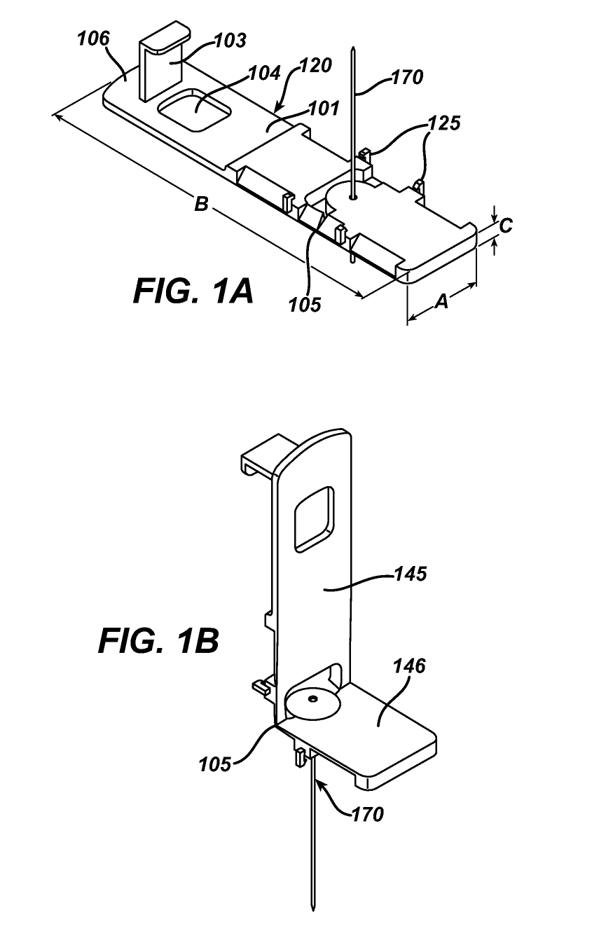

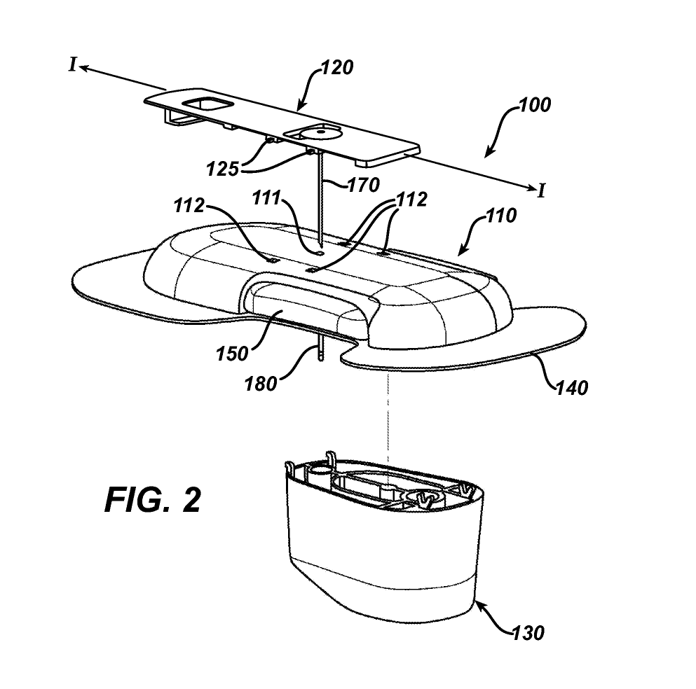

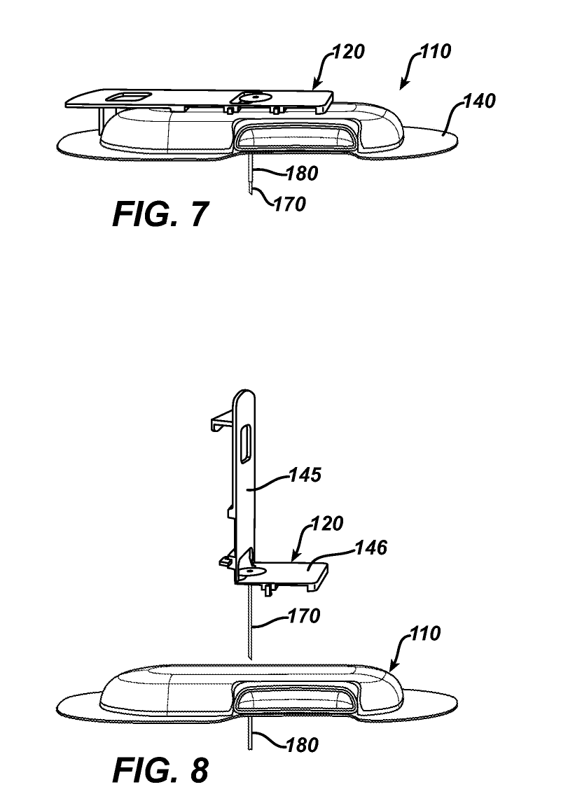

In FIGS. 1, 1A, 1B, 2, and 7 is shown an infusion system 100 of the invention with infusion device 110, needle handle 120 that carries insertion needle 170, and cannula cover 130. Optionally and preferably an adhesive layer 140 is on the underside of the device. Actuators 150 and 160 are provided on either side of the device. Also, as shown in FIG. 2, the outer shell of device 110 includes a needle opening 111 to receive needle 170 and one or more notches 112 to receive and releasably join the corresponding latching post 125 of needle handle 120 to device 110. Optional opening 104 is also shown in needle handle 120, which opening may facilitate handling as well as decrease the amount of material needed for handle 120.

The needle handle of the invention has a low profile meaning that the handle height is comparatively less than either or, and more preferably, both the handle's width and length. In one embodiment, and as shown in FIGS. 1, 1A and 1B, handle 120 has elongate body 101 with a width A and length B either of which is greater than height C.

Handle body 101 is releasably affixed to the body 110 of the infusion device by any suitable means such as mechanical including catches, posts, clasps, latches, tabs, hooks, interference fits and the like, chemical such as an adhesive, solvent bond, thermal weld, ultrasonic weld, laser weld, and the like, or a combination thereof. Thus, handle 120 provides a resistive force against the pressure exerted on the cannula and needle as device 110 is inserted onto the user's skin and the needle and cannula into the user's tissues. In the embodiment shown in FIG. 1, multiple mechanical attachments are used. Handle 120 has catch 103 extending downwardly from bottom surface 106 of body 101. For purposes of the invention, “upward” means above the plane I-I shown in FIG. 2. “Downward” means below the plane of I-I. As shown, catch 103 is “L” shaped and is provided at one end 107 of handle 120, but such a catch may be of any suitable size and shape provided it is suitable to releasably engage one or both of the side and underside of device 110. Optionally and preferably, one or more latching posts 125 extend downwardly from bottom surface 106 or the sides of handle 120 and are releasably received in notches 112 of device 110. Feet 125 engaging notches 112 and catch 103 engaging body 110 serve to releasably hold handle 120 in place in its non-deployed position. Also extending downwardly from bottom surface 106 is insertion needle 170 affixed to the handle via adhesive bond, solvent bond, ultrasonic bond, press fit, or swaged to the needle handle, and the like, which needle extends into device 110.

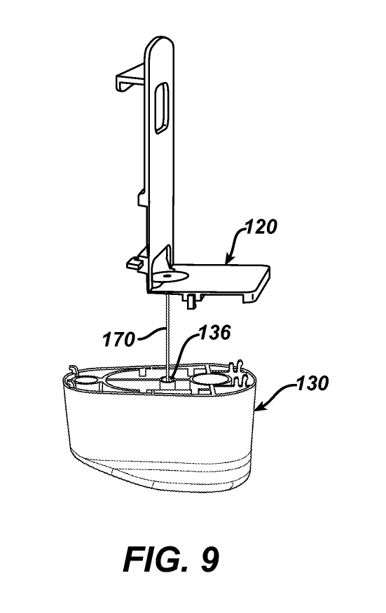

Driving device 110, whether manually or with the aid of an inserter, onto the user's skin inserts needle 170 and cannula 180 into the user's tissues to the desired depth. Needle handle 120 is then used to extract needle 170 from the device. To deploy handle 120, the user pulls upwardly on end 107 releasing catch 103. The upward movement of end 107 exerts a pivoting force on the remainder of body 101 and causes it to flex upwardly at hinge 105. The hinge may be any suitable hinge for permitting the desired flexion of the handle. Preferably, the hinge is a molded or a “living” hinge that is formed by creating an area of material that is thinner in thickness as compared to the thickness of the portions of the handle immediately adjacent the hinge. As shown, handle first portion 145 and second portion 146 are on either side of hinge 105 and are of a thickness greater than hinge 105. The flexing movement also results in extraction of all or several of posts 125 from notches 112 and formation of handle 120 into an “L”-shaped handle configuration as seen in FIGS. 1B and 8. The user then uses the upwardly extending portion of the “L”-shaped handle to pull upwardly extracting any remaining posts 125 from notches 112 and needle 170 from the device and detaching needle handle 120, along with needle 170, from the device while maintaining the position of cannula 180 within the user's tissues. Needle 170 may be disposed of by attaching it to cannula cover 130 as shown in FIG. 9, in which is depicted needle 170 being received into elongated cavity 136 of cover 130.

The needle handle may be formed from any suitable material capable of having a living hinge formed therein and that is sufficiently strong and rigid to accomplish needle extraction. Suitable such materials include polymers with a Young's Modulus from about 150 to about 450 kpsi, tensile strength in the range of about 4,000 to 15,000 psi, and flexural modulus in the range of about 100 to about 500 kpsi. Preferably the handle is formed from polyethylene, polypropylene, nylon, polyvinyl chloride or the like, or combinations thereof.

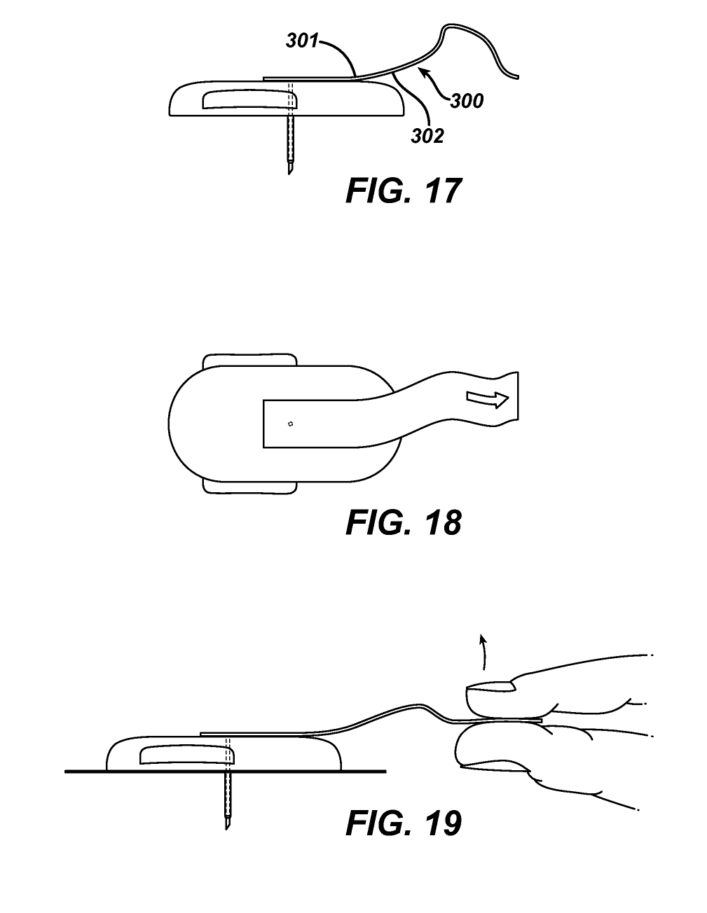

In an alternative embodiment, needle handle 120 may be formed of a flexible material such as a membrane, film or the like. As shown in FIGS. 17 through 19, the body 301 of handle 300 is formed of a flexible material. Suitable materials are those that can be formed into thin films, are bondable, tear resistant, and non-elastic. Exemplary materials include polyethylene terephthalate, polyethylene terephthalate glycol, polyvinylidene fluoride, a polyimide film such as KAPTON™, polycholorotrifluoroethylene, and the like and combinations thereof. Handle 300 is releasably affixed to the infusion device by use of an elastic, releasable adhesive, similar to that used on skin attaching devices, on the underside 302 of handle body 301. Alternatively, no adhesive or attachment may be required to hold body 301 onto device 110. Rather, friction between the septum and cannula may hold handle 301 in place.

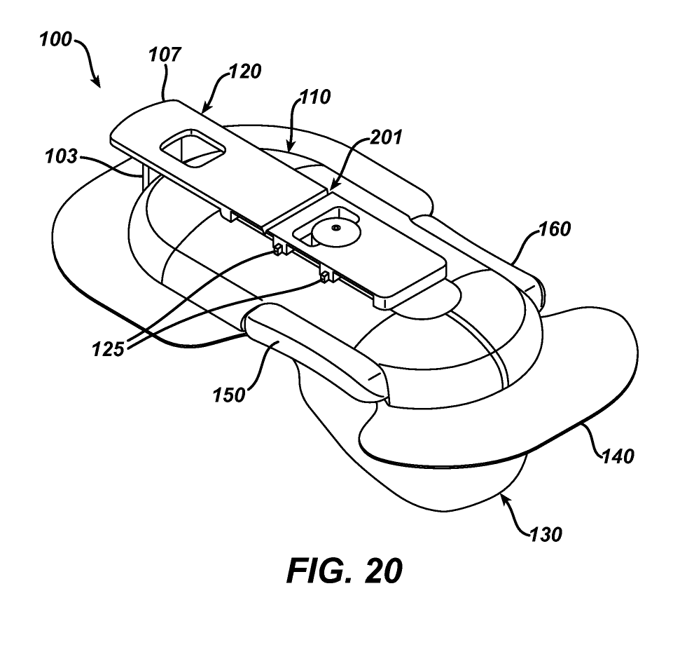

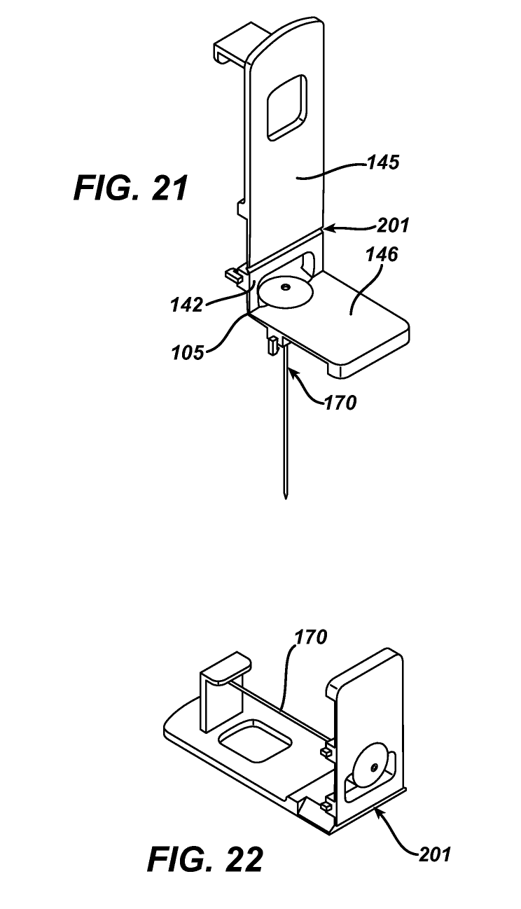

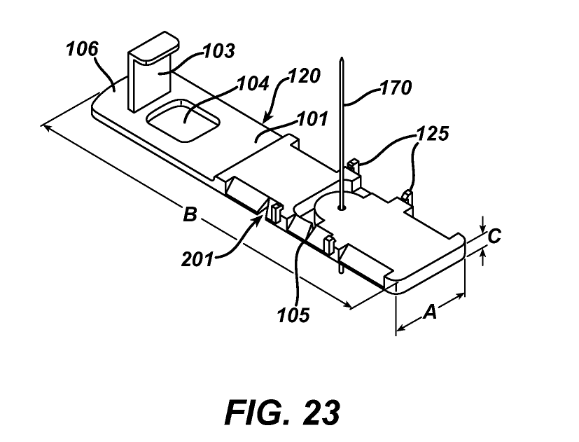

Another alternative is shown in FIGS. 20 through 23 in which handle 120 includes a second hinge 201. Second hinge 201 is formed between first handle portion 145 and a third handle portion 142, which third portion is itself a part of first portion 145 and is bounded by hinge 201 and hinge 105. After deployment of the handle (FIG. 21) and removal of the needle from device 110, first handle portion 145 is returned to its original, flat orientation adjacent second portion 146. A part of first handle portion 145 is then folded downwardly at hinge 201 to form a protective enclosure for the end of needle 170 as shown in FIG. 22. The handle and needle then may be safely disposed.

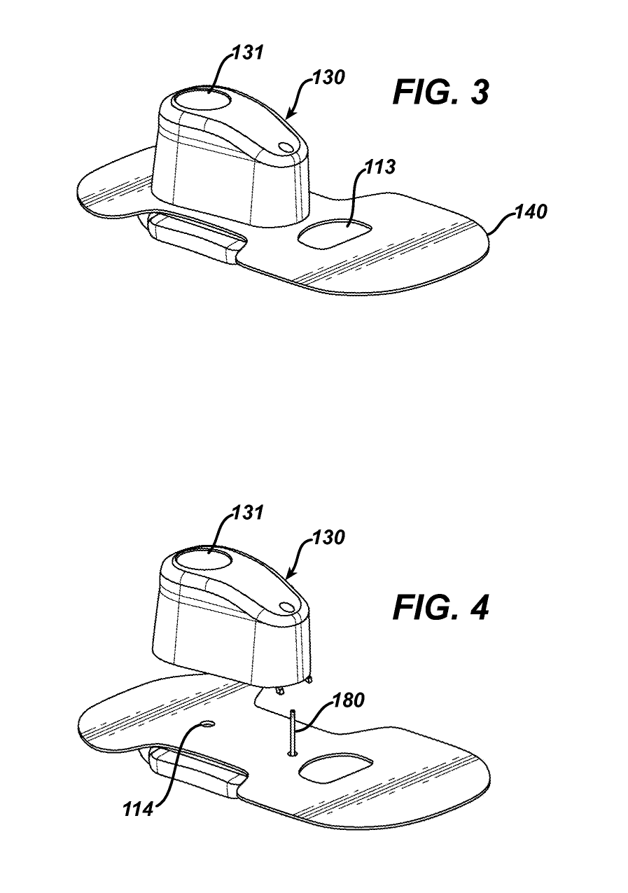

Referring to device 110, as shown in FIG. 3, adhesive layer 140 that, as shown, includes optional viewing window 113, and cannula cover 130 are located on the underside, or user skin facing side, of device 110. Adhesive layer 140 is configured to be attached to the user's skin and includes a cover, not shown, that is removable prior to adhering the adhesive layer 140 to a user's skin. Although viewing window 113 is shown located on the underside of the device, it may be located on the top of the device. Window 113 is a clear or transparent portion that enables the user to view the contents of the device's reservoir including any bubbles introduced during filling of the device.

Cannula cover 130 has opening 131 configured to receive a syringe and needle. Optionally, opening 131 may include a cover, not shown. Port 114 on the underside of device 110, and shown in FIG. 4, is in line with opening 131 when cover 130 is attached to the device. Opening 131 through port 114 ultimately communicates with the device's reservoir. Cover 130 guides the coupling of a syringe and also serves as a protective cover for cannula 180.

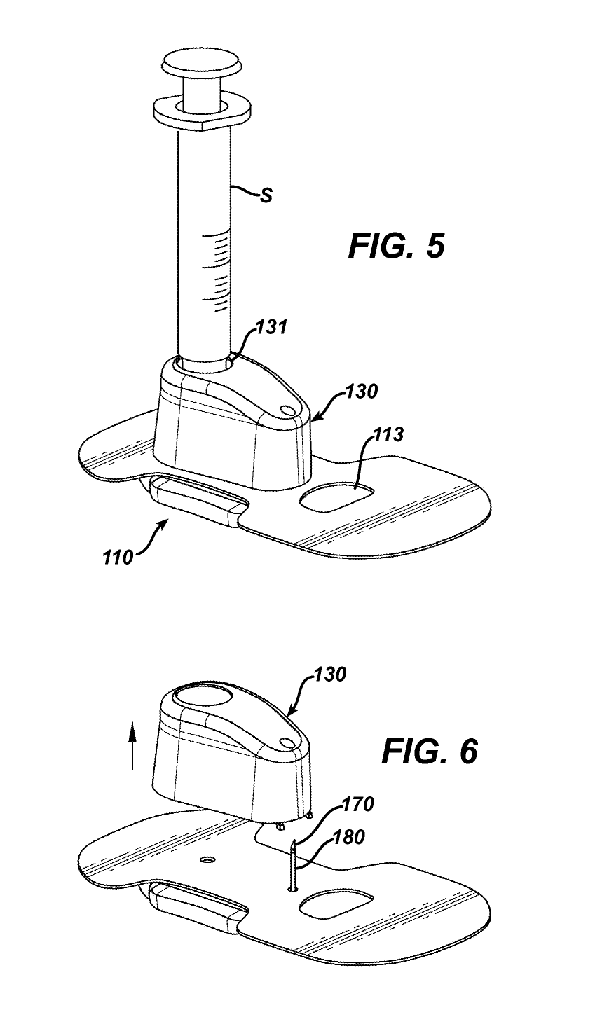

In FIG. 5 is illustrated the filling of device 110 by insertion of syringe S into opening 131. Once the device's reservoir is filled, and the air removed from the reservoir and fluid pathways by operating the pump buttons, cannula cover 130 is removed from the device as shown in FIG. 6 to expose cannula 180 and needle 170. In one embodiment, a cover on the adhesive layer 140 may be attached to cannula cover 130 so that, when cover 130 is removed, the adhesive layer cover is also removed. The device is then driven onto the user's skin by the user pressing the device onto the skin or through the aid of an inserter that mechanically drives the device onto the user's skin.

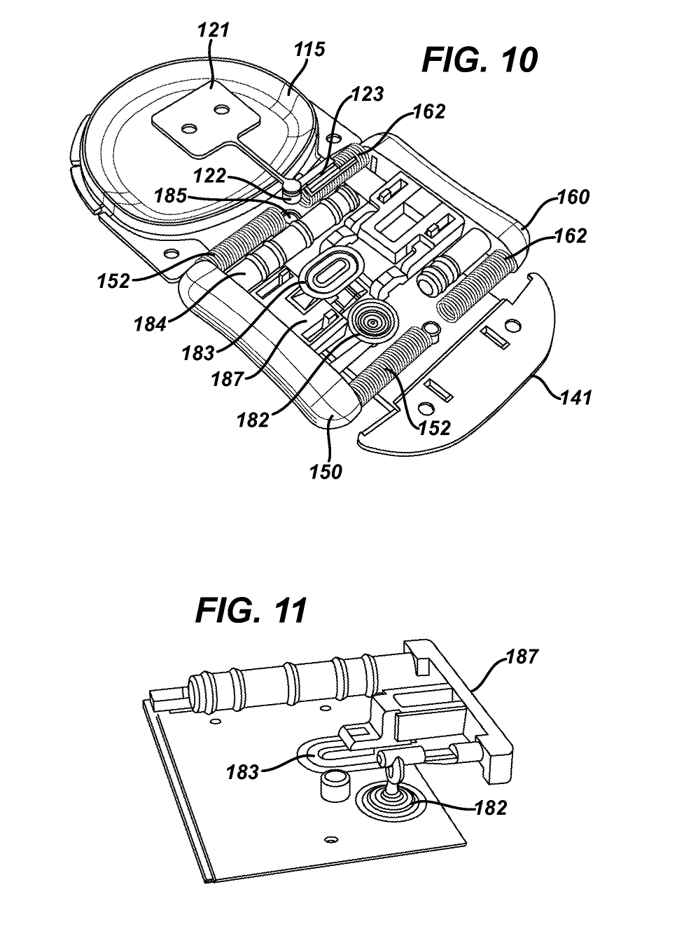

The internal components for one embodiment of the invention are shown in FIG. 10, which is a top perspective view with the top surface of device 110 removed. Shown is reservoir 115, needle hole cover 121, needle septum 122, septum pincher 123, actuator buttons 150 and 160, actuation springs 152 and 162, base plate 141, valve stem 184, locking mechanism 187, and cannula port 185. Reservoir 115 may be a hollow base overlaid with one or more layers of flexible, bio-compatible film. The film may be a laminate of sufficient thickness and flexibility and chemical characteristics to hold the desired medication. The film may be in the form of a fillable bag or pouch overlaid on the base or it may be in the form of a flexible cover for the base with the medication directly introduced between the film and the base.

In accordance with this embodiment, actuation occurs by manual depression of actuator buttons 150 and 160, which causes the medication to flow through the pump mechanism and around the valve stem 184 and, ultimately, through the cannula and into the user. The pump mechanism is configured to be acted on the buttons so that, when the actuators are activated, the forward actuation stroke propels medication from the pump mechanism into the cannula. The return stroke action then pulls medication from the reservoir pump into the pump mechanism for delivery upon the next actuation.

Optionally and preferably, the pump includes a last-dose lock-out mechanism 182, as shown in FIG. 11, that detects the absence of medication flowing from the reservoir to the pump mechanism by means of a pressure drop that acts on a deflectable diaphragm. In such absence, mechanism 182 engages a portion of the locking mechanism 187, which locking mechanism 187 is connected to actuator button 150. When engaged by the last-dose lock-out mechanism 182, mechanism 187 prevents the activation of button 150.

Also optionally and as shown in FIG. 11, the pump may include an occlusion detection mechanism 183 that detects occlusions within the medication flow stream through the pump mechanism by means of high pressure due to blocked flow. On detection of an occlusion, mechanism 183 engages a portion of locking mechanism 187 which, in turn, engages a portion, and prevents activation, of actuator button 150.

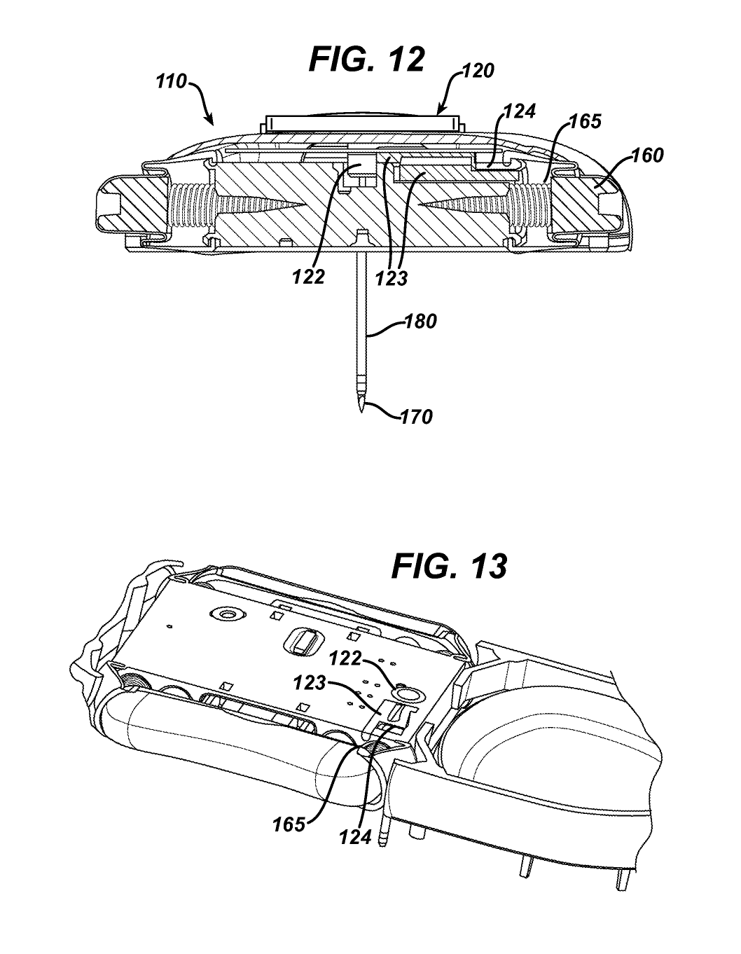

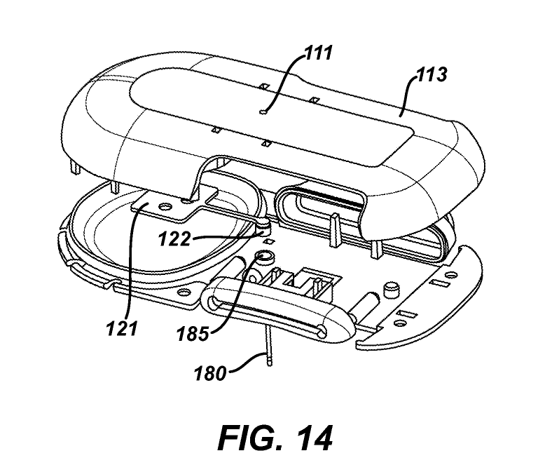

The device also includes a closable needle septum, as shown in FIGS. 12, 13 and 14. In order to maintain the sterility and integrity of the internal fluid pathway after withdrawal of needle 170, needle septum is located on the inside of the device and is configured to be a self-sealing septum to prevent materials from entering the device, or drug escaping from the device, after withdrawal of needle 170. In the event withdrawal needle 170 from septum 122 results in a hole in the septum, septum pincher 123 is provided to press the septum closed during button actuation. This prevents seepage of medication. As can be seen in FIG. 12, pincher 123 is slidable within a cavity 124 within the device. Pincher 123 is configured to be acted on by portion 165 of actuator 160. Actuator 160 being depressed causes pincher 123 to slide along cavity 124 to contact and press septum 122 and collapse it around any opening left by the needle. In one embodiment, after actuation, when actuator button 160 returns to its normal position, pincher 123 also slides back to its original position within cavity 124. Alternatively, pincher 123 may be configured to permanently press against the septum on first actuation such as by providing a latching portion on the pincher that corresponds to a latch-receiving portion in the cavity wall.

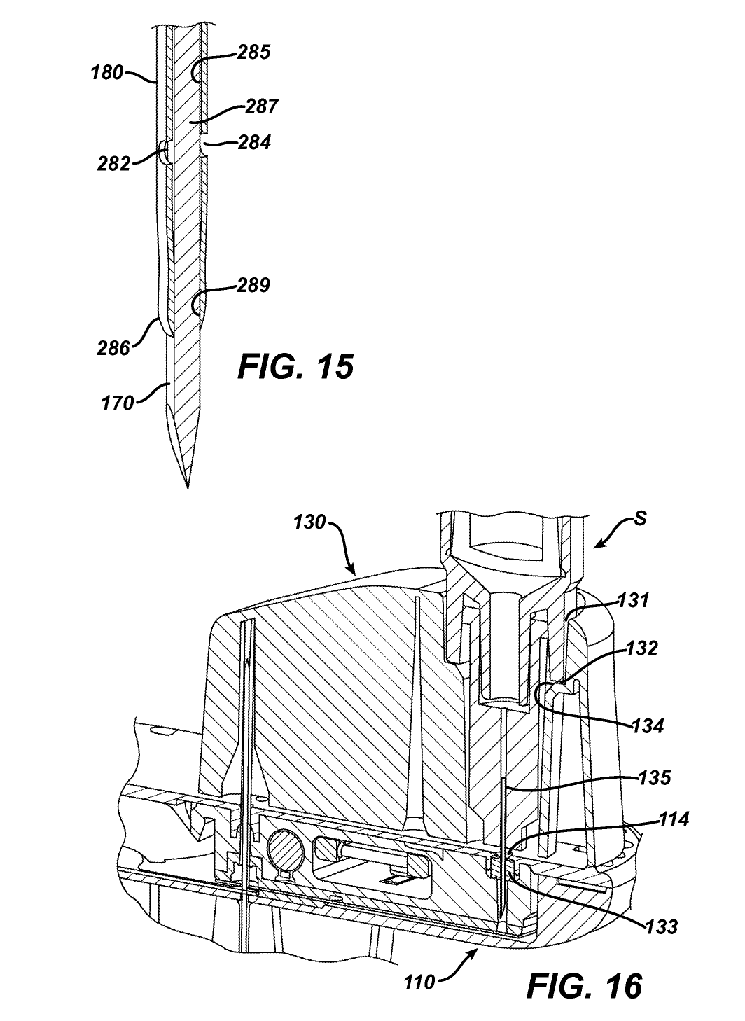

In FIG. 15 is shown cannula 180 and an embodiment of insertion needle 170 in which cannula 180 has aligned side ports 282 and 284, preferably directly opposite each other to deliver medication in opposite directions. The inner channel of cannula 180 is preferably tapered ay distal end 286 and terminates in a central, tip end, output port 289 the size of which port is such that it is nearly sealed by insertion needle 170 while annular passage 287 is provided with two side ports 282 and 284 due to the taper in inner channel 285. During priming, nearly all of fluid is forced down annular passage 287 and exits through side ports 282 and 284.

FIG. 16 shows the syringe S in the filing position within device 110. An annular surface 134 on syringe S engages annular shoulder 132 of filling channel 131 and forms a stop that limits the depth of penetration of syringe needle 135 within fill port 114. The tip end of syringe needle 135 is permitted to extend only through the filling septum 133 and, thus, protects the device from damage.

What is claimed:

1. A device, comprising:
an infusion device having a reservoir therein;
a low profile handle releasably affixed to the infusion device; and
an insertion needle extending from one side of the needle handle and into an opening in the infusion device.

2. The device of claim 1, wherein the handle further comprises a first hinge between a first and a second portion of the handle, the hinge permitting upward flexion of the first handle portion with respect to the second handle portion.

3. The device of claim 2, wherein the handle further comprises a second hinge on the first handle portion, the second hinge permitting flexion of at least a part of the first handle portion downwardly with respect to the second portion.

4. The device of claim 1, 2, 3, or 4, wherein the handle is comprised of a polymer having a Young's Modulus of about 150 to about 450, a tensile strength of about 4,00 to about 15,000 psi, and a flexural modulus of about 100 to about 500 kpsi.

5. The device of claim 1, 2, 3, or 4, wherein the handle comprises polyethylene, polypropylene, nylon, polyvinyl chloride, or combinations thereof.
